(19) United States
(12) Patent Application Publication
(10) Pub. No.: US 2005/0015846 A1
(43) Pub. Date: Jan. 27, 2005

(54) Polyvinyl chloride article having improved durability

(75) Inventors: Maris Vistins, Alpharetta, GA (US); Scott Stephen Englebert, Fairburn, GA (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/606,616

(22) Filed: Jun. 26, 2003

(51) Int. Cl. A41D 19/00; A61F 9/00; A42B 1/00

(52) U.S. Cl. 2/161.7

(57) ABSTRACT

A method of forming a glove having improved fingertip puncture resistance is disclosed. The method includes providing a glove former, the former pivotably attached to a chain assembly, dipping the former into a plastisol in a first position, the position being substantially vertical, removing the former from the plastisol, pivoting the former to a second position, the second position forming an angle less than 90 degrees with respect to the first position, and maintaining the former at the second position until the plastisol forms a gel on the former.

### BACKGROUND

In recent years, there has been an increasing emphasis in the medical community on developing gloves that offer various degrees and types of protection. Medical practitioners are frequently exposed to sharp objects that may puncture the glove and may compromise the barrier afforded by the glove. As such, there is a recognized need for a glove with improved resistance to puncture. The most common area of failure in a glove due to puncture is the fingertip. Failure in the fingertip area may lead to health hazards such as abrasions, cuts, infection, and contamination by hazardous materials. As such, there is a need for a glove that has improved puncture resistance in the fingertip area.

Gloves formed from thermoplastic resins, such as polyvinyl chloride (PVC), have a history of poor fingertip durability in use relative to gloves formed from a coagulated rubber latex. This disparity is caused by inherent differences in the materials used to form the gloves. A glove formed from a coagulant-based dipping process typically has fingertips that have a thickness greater than that of the rest of the glove because both the first and last point of contact between the coagulant on the glove former and the latex is the fingertip, and the latex begins to coagulate immediately upon contact with the coagulant on the former. Gloves formed from a plastisol, such as a PVC plastisol, generally suffer from deficient fingertip thickness because the plastisol does not thicken or gel until the plastisol is exposed to heat at a specific gel temperature, so the plastisol tends to continuously drain from the former until the former is exposed to sufficient heat.

One potential solution to this problem would be to increase the thickness of the entire glove, including the fingertips. However, a thicker glove may diminish the user's sense of touch and therefore be less desirable.

### SUMMARY OF THE INVENTION

The present invention generally relates to a method of forming a glove having improved fingertip puncture resistance. The method includes providing a glove former that is pivotably attached to a chain assembly, dipping the former into a plastisol in a substantially vertical first position, removing the former from the plastisol, pivoting the former to a second position that forms an angle less than 90 degrees with respect to the first position, and maintaining the former at the second position until the plastisol forms a gel on the former. The second position may form any suitable angle with respect to the first position, and in some instances, the second position may form an angle of from about 60 degrees to about 85 degrees with respect to the first position. In other instances, the second position may form an angle of from about 70 degrees to about 83 degrees with respect to the first position. In yet other instances, the second position may form an angle of from about 75 degrees to about 80 degrees with respect to the first position. The former may be heated while being maintained in the second position.

The present invention also relates to a polyvinyl chloride glove having improved fingertip puncture resistance. The glove includes a palm portion having a palm thickness, and a plurality of fingers extending from the palm portion, each finger having a fingertip distal to the palm portion, where the fingertip has a fingertip thickness substantially equal to the palm thickness. In some instances, the fingertip thickness may be from about 0.1 mm to about 0.2 mm. In other instances, the fingertip thickness may be from about 0.11 mm to about 0.15 mm. In another instance, the fingertip thickness may be about 0.12 mm. The glove may be formed by providing a glove former, the former pivotably attached to a chain assembly, dipping the former into a polyvinyl chloride resin plastisol in a first position, the position being substantially vertical, removing the former from the plastisol, pivoting the former to a second position, the second position forming an angle less than 90 degrees with respect to the first position, and maintaining the former at the second position until the plastisol gels on the former.

The present invention also relates to a method of determining fingertip puncture resistance in a glove. The method includes preparing a glove fingertip sample, placing the sample onto a cylindrical sample mount, advancing a probe toward the sample, contacting the probe to the sample, and measuring the force required to perforate the sample. The thickness of the sample may be measured where desired. In some instances, the probe may be advanced toward the sample at from about 100 mm/min to about 800 mm/min. In other instances, the probe may be advanced toward the sample at from about 400 mm/min to about 600 mm/min.

### BRIEF DESCRIPTION OF THE DRAWINGS

FIG. 1 depicts an exemplary article that may be formed according to the present invention.

FIG. 2 depicts an exemplary glove formation process.

FIG. 3 is a schematic cross-sectional illustration of the article of FIG. 1 taken along a line 3-3.

FIG. 4 depicts the glove formation process according to the present invention.

FIG. 5 depicts an exemplary test apparatus according to the present invention for determining the puncture resistance of a glove fingertip.

### DESCRIPTION

The present invention generally relates to a method of forming a glove having increased puncture resistance in the fingertip, and a glove formed from such a method. The method of the present invention generally results in a glove having a fingertip thickness that is substantially equal to the thickness of other parts of the glove, for example, the palm, without having to increase the thickness of the entire glove. As used herein, a “substantially equal” thickness refers to a thickness that is within 0.05 mm of another thickness, as measured by any suitable device such as a caliper, as described herein. The fingertip thickness is increased by adjusting the angle of the former during gelation of the PVC plastisol. This increase in fingertip thickness results in an improved puncture resistance, as is described herein. The present invention further relates to a method of determining puncture resistance of a glove fingertip.

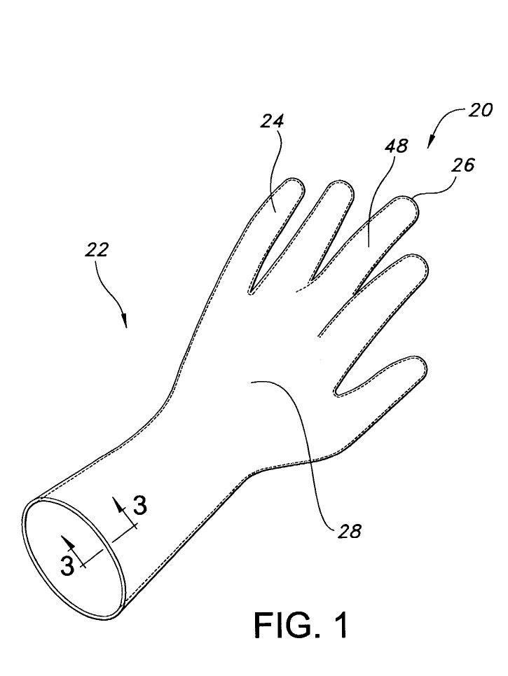

As depicted in FIG. 1, a glove 20 formed according to the present invention generally includes a palm portion 22 and a plurality of fingers 24. The fingers 24 extend from the palm portion 22. Each finger 24 of the glove 20 has a fingertip 26 distal to the palm portion 22. The fingertip 26 has a thickness (“fingertip thickness”) substantially equal to the thickness of the palm portion (“palm thickness”).

The fingertip 26 may generally have a fingertip thickness greater than about 0.10 mm, as measured using any suitable technique or device, such as a caliper, as described herein. In some embodiments, the fingertip 26 may have a fingertip thickness of from about 0.10 mm to about 0.20 mm. In other embodiments, the fingertip 26 may have a fingertip thickness of from about 0.11 mm to about 0.15 mm. In yet other embodiments, the fingertip 26 may have a fingertip thickness of about 0.12 mm.

Thus, in some embodiments, the palm portion 22 may have a palm thickness of from about 0.10 to about 0.20 as measured generally in the center 28 of the palm portion 22. In other embodiments, the palm portion 22 may have a palm thickness of from about 0.11 mm to about 0.15 mm. In yet other embodiments, the palm portion 22 may have a palm thickness of about 0.12 mm.

The glove of the present invention may be formed using a variety of processes, for example, dipping, spraying, tumbling, drying, and curing. An exemplary dipping process for forming a glove is described herein, though other processes may be employed to form various gloves having different characteristics. Furthermore, it should be understood that a batch, semi-batch, or a continuous process may be used with the present invention.

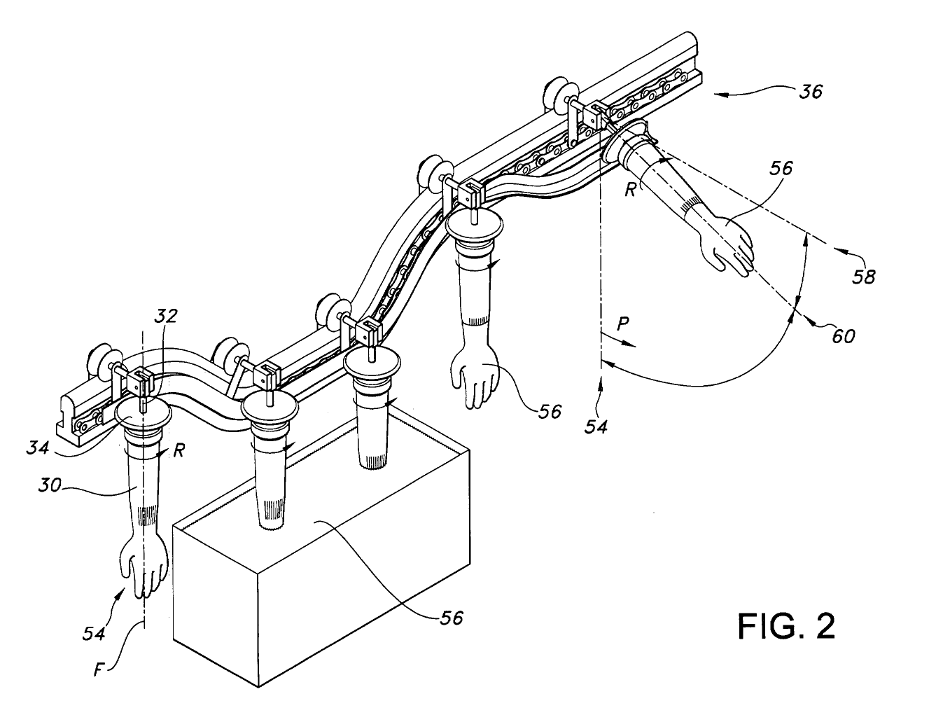

As depicted in FIGS. 1 and 2, a glove 20 is formed on a hand-shaped mold, termed a “former” 30. The former 30 may be made from any suitable material, such as glass, metal, porcelain, or the like. The surface of the former defines at least a portion of the surface of the glove 20 to be manufactured. The former 30 is generally attached to a carrier 32 by a bearing 34, so that the former 30 is capable of rotating in a direction R about an axis F along the length of the carrier 32. The carrier 32 is pivotably attached to a chain assembly 36 that is advanced through various stages of the glove formation process. The carrier 32 is capable of pivoting in a direction P that is perpendicular to the length of the chain assembly 36.

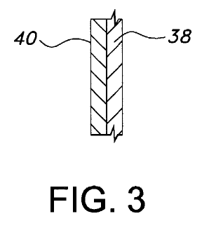

In general, the glove is formed by dipping the former into a series of compositions as needed to attain the desired glove characteristics. The glove may be allowed to solidify between layers. Any combination of layers may be used, and although specific layers are described herein, it should be understood that other layers and combinations of layers may be used as desired. Thus, in one embodiment, the glove 20 may include a substrate body 38 and a donning (wearer-contacting) layer 40 (FIG. 3).

In one embodiment, the substrate body may be formed from a plastisol using a dipping process. As used herein, a “plastisol” refers to a dispersion of fine resin particles in a plasticizer. The plastisol is formed by mixing the resin particles into the plasticizer with sufficient shear to form a stable system. Any suitable resin may be used as desired, and in some instances, the resin includes polyvinyl chloride (PVC). While articles formed from PVC are described in detail herein, it should be understood that any other suitable thermoplastic material or combination of thermoplastic materials may be used with the present invention. Thus, for example, the resin may include a styrene-ethylene-butylene-styrene block copolymer, a nitrile butadiene polymer, or any other polymer capable of forming a film without use of a coagulant. Furthermore, while exemplary process conditions are described herein, it should be understood that such conditions depend on the desired thickness of the article, the viscosity of the composition, the time required to gel the article, and so forth.

The former may first be heated to a temperature of about 100° F. (38° C.) to about 200° F. (93° C.), for example, 150° F. (66° C.). The former is then dipped into a plastisol 56 containing a suitable thermoplastic resin, for instance, PVC, a plasticizer, and a heat stabilizer (FIG. 2). The composition may be maintained at any suitable temperature, and in some instances, is maintained at a temperature of from about 75° F. (24° C.) to about 175° F. (79° C.), for example, 105° F. (40° C.). This dipping generally occurs in a substantially vertical first position 54 as shown in FIG. 2.

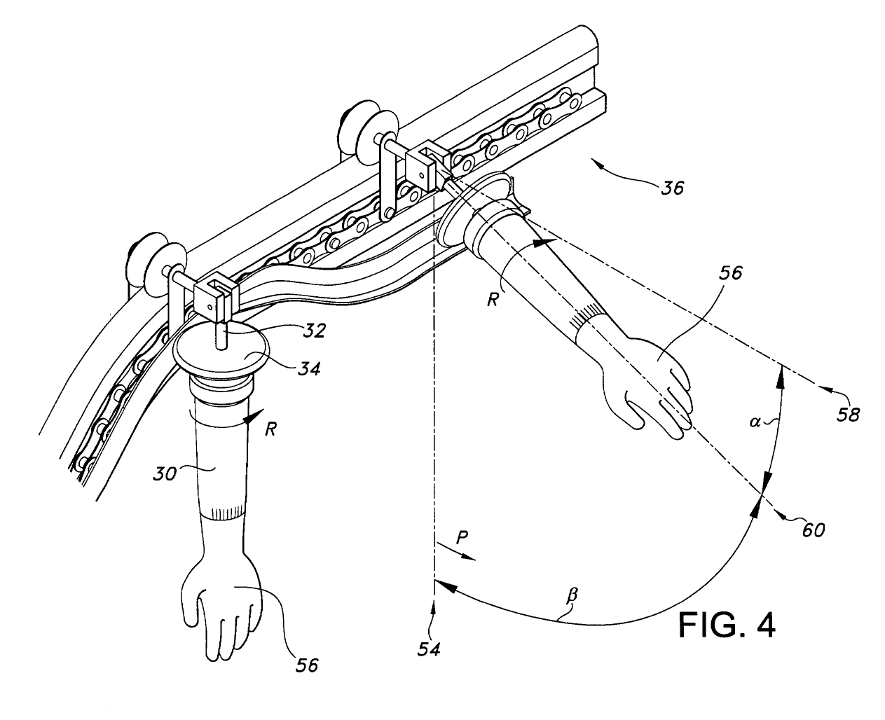

The formers are then removed from the composition to drain. The time permitted to drain (“drain time”) determines the mass of the glove, its thickness, and so forth, based on the temperature of the former and the viscosity of the plastisol. Typical PVC glove formation processes permit the former 30 to drain for a specified amount of time, and then pivot the former upward in a direction P to a horizontal position 58 where it rotates in a direction R until the plastisol 56 gels on the former 30 (FIG. 4). While rotating the former, the plastisol on the former is exposed to heat to cause the PVC to gel. At this point, the PVC no longer flows and the base structure of the glove is established. The formers are then advanced through a fusion oven where the substrate body is permitted to fuse on the former. In one instance, the fusion oven may be maintained at about 300° F. (149° C.) to about 500° F. (260° C.), for example, 450° F. (232° C.), and the former may be in the oven for about 3 to about 8 minutes, for example, 6 minutes.

It has been discovered, however, that this typical practice of pivoting the former to a horizontal position and advancing it to the fusion oven is largely responsible for insufficient finger tip thicknesses. As such, the present invention contemplates pivoting the formers to a second position 60 that is less than horizontal to enable the plastisol to flow to the fingertip and accumulate the desired thickness as the plastisol 56 gels (FIG. 4). The minimum and maximum angle β formed between the first position and the second position depends largely on the temperature of the former and the viscosity of the plastisol. Furthermore, the time required to gel the plastisol depends on the temperature of the fusion oven and the dwell time within the oven.

In general, as the viscosity of the plastisol increases, less of a deviation from horizontal is required because less flow to the finger tips is needed to accumulate the desired thickness. As the temperature of the fusion oven increases, less deviation from a horizontal position is required because the plastisol will gel more quickly and less flow is needed. Thus, for a given oven temperature and a given plastisol viscosity, the angle may need to be adjusted to arrive at the desired glove and finger tip characteristics.

In some embodiments, the former 30 may be pivoted to a second position that forms and angle α of from about 5 degrees from horizontal to about 30 degrees from horizontal. In other embodiments, the former 30 may be pivoted to a second position that forms an angle α of from about 7 degrees from horizontal to about 20 degrees from horizontal. In yet other embodiments, the former 30 may be pivoted to a second position that forms an angle α of from about 10 degrees from horizontal to about 15 degrees from horizontal.

Thus, the glove of the present invention may be formed by providing a glove former, the former pivotably attached to a chain assembly, dipping the former into a plastisol in a substantially vertical first position, removing the former from the plastisol, pivoting the former to a second position, the second position forming an angle less than 90 degrees with respect to the first position, and maintaining the former at the second position until the plastisol forms a gel on the former.

Likewise, the second position may form any desired angle β with respect to the first position, and in some embodiments, the second position forms an angle β of from about 60 degrees to about 85 degrees with respect to the first position. In other embodiments, the second position forms an angle β of from about 70 degrees to about 83 degrees with respect to the first position. In yet other embodiments, the second position forms an angle β of from about 75 degrees to about 80 degrees with respect to the first position. It should be understood that while various ranges are set forth herein, that exact positions depend on process conditions, and that such positions are contemplated by the present invention.

The fused PVC on the former is then cooled to a temperature of about 100° F. (38° C.) to about 200° F. (93° C.), for example, 150° F. (66° C.), by exposing the formers to one or more cooling fans, blowers, or water sprays as appropriate.

Where desired, the former may be dipped into a composition to form a donning layer to facilitate donning of the glove. The donning layer composition may be maintained at about 100° F. (38° C.) to about 200° F. (93° C.), for example, 150° F. (66° C.). The donning layer on the former may then be dried in an oven, for example, for about 2-3 minutes at a temperature of about 200° F. (93° C.) to about 400° F. (204° C.), for example, 300° F. (149° C.).

The donning layer may be formed from any suitable polymer, and in some embodiments, may be formed from a polyurethane. One such polyurethane that may be suitable for use with the present invention is available from Soluol Chemical Co., Inc. (West Warwick, R.I.) under the trade name SOLUCOTE® 117-179. SOLUCOTE® 117-179 is provided as a waterborne polyurethane dispersion having from about 10-20 mass % total solids content (TSC). In other embodiments, the donning layer may be formed from an acrylic polymer. One such acrylic polymer that may be suitable for use with the present invention is available from Jatrac, Inc. (Kyoto, Japan) under the trade name SMOOTHER Anti-Stick Agent.

While exemplary donning layer materials are set forth herein, it should be understood that any suitable donning layer material may be used as desired. Furthermore, various lubricating materials may be added to the donning layer composition as desired or needed to enhance donning. Some such materials may include a flattening agent, a lubricant, for example, a wax or a silicone, or particulate matter, for example, silica.

The former is then sent to a bead rolling station, where the cuff is rolled slightly and permitted to solidify. The former may then be transferred to a stripping station where the glove is removed from the former. The stripping station may involve automatic or manual removal of the glove from the former. For example, in one embodiment, the glove is manually removed and turned inside out as it is stripped from the former. By inverting the glove in this manner, the donning layer formed on the exposed surface of the substrate body on the former becomes the interior of the glove.

The present invention further contemplates a method of determining the resistance of a glove fingertip to puncture. The method measures the force required to puncture a glove at the fingertip and may be used to predict actual use conditions. While a detailed description of the test method is provided herein, it should be understood that variations on the procedure are also contemplated by the present invention.

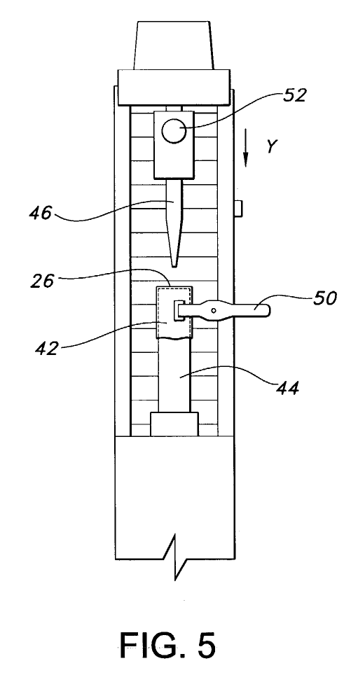

As depicted in FIG. 5, the method generally includes preparing a sample 42 from a glove fingertip, placing the sample 42 onto a cylindrical sample mount 44, driving a probe 46 toward the sample 42, contacting the probe 46 to the sample 42, and measuring the force required to puncture the sample 42. The thickness of the sample may generally be measured prior to testing so that the relationship between the glove thickness and the resistance to puncture may be determined.

The sample 42 is prepared by cutting a specified length from a finger of a glove to be evaluated. If desired, the test method may specify a particular glove finger to be used, for example, the middle finger 48 (FIG. 1). Any suitable length may be removed, and in some instances, a length of from about 30-45 mm, may be removed. In some instances, a length of about 38 mm may be removed.

The thickness of the sample is then measured if desired. The thickness may be measured multiple times to obtain an average where desired, for example, 3 times. Any suitable device may be used to measure the thickness of the sample, for example, a caliper. The specifications for the caliper used may be as follows: measuring range of 0 to 12.7 mm; accuracy at 20° C. of 0.02 mm; measuring force of 1.4N or less; stem diameter of 9.525 mm; and contact point of 4.1 mm.

The sample 42 is then placed on the sample mount 44, which may have a cylindrical shape as depicted in FIG. 5. The sample mount 44 may be made from any suitable material, and in some instances, the sample mount 44 is made from stainless steel. Where desired, a clamp 50 may be provided to secure the sample 42 on the sample mount 44. When the sample is fully mounted and any wrinkles have been manually removed by adjusting the sample on the mount, the sample is ready to be tested. In some instances, it may be desirable to lightly dust the sample with a powder, such as talc, to ensure that the probe does not stick to the sample during puncture. Where such sticking does occur, the perforation may be artificially larger than a perforation that might occur during actual use. While no specific amount is required, a light dusting on the sample may suffice to eliminate any concerns about inaccurate perforation size.

When the sample 42 has been fully prepared, the probe 46 may be advanced toward the sample in a direction Y to determine the resistance of the glove fingertip 26 to puncture. Depending on the test apparatus used, the probe may be mounted on a cross head 52 or other suitable mounting means. The probe may be made of any suitable material, and in some instances, made from precision cut stainless steel. The probe may advance toward the sample at any desired rate, and in some instances, the probe may advance toward the sample at from about 100 mm/min to about 800 mm/mm. In other instances, the probe may be driven toward the sample at from about 300 mm/mm to about 700 mm/min. In yet other instances, the probe may be driven toward the sample at from about 400 mm/mm to about 600 mm/min. In still other instances, the probe may be driven toward the sample at about 500 mm/min.

As the probe 46 contacts the sample 42, the force required to puncture the fingertip 26 is measured. Any suitable device may be used to measure the force, such as a constant rate of extension tensile tester. The data may be recorded using a computer-based data acquisition and frame control system (not shown).

The method of the present invention has been found to accurately represent actual use conditions. Prior to the method of the present invention, the only accepted means of evaluating puncture resistance of a glove was ASTM F1306-90 (“ASTM”), entitled “Slow Rate Penetration Resistance of Flexible Barrier Films and Laminates”. In general, the ASTM measures the puncture resistance of the specimen by clamping the sample in a universal tester and driving a probe into contact with the sample at a fixed velocity until the sample perforates, i.e., until the sample develops a visible flaw. According to the ASTM procedure, a 76 mm by 76 mm specimen is prepared. The thickness is then measured three times in the center of the specimen and averaged. The specimen is then placed on a specimen clamping fixture. The cross head speed of the universal tester is adjusted to 25 mm/min. The probe is then driven into the center of the specimen until it perforates, and the force required to perforate the film is recorded.

While the ASTM provides a relative measure of slow puncture resistance for glove samples, it is unable to accurately predict the resistance of a glove fingertip to puncture. First, the sample size required by the ASTM is too large to cut a sample from a glove fingertip, which generally measures about 20 mm by about 20 mm. Thus, the only portion of the glove that can be used with the ASTM is the palm portion. Furthermore, the slow rate used by the ASTM does not accurately represent the type of punctures that occur in the fingertip area because such punctures are-generally caused by rapid contact of the donned glove to a sharp object.

These discoveries are evidenced by the following examples, which are not intended to be limiting in any manner.

### EXAMPLE 1

Commercially available glove samples were evaluated for puncture resistance according to the test method of the present invention.

A Constant-Rate-of-Extension (CRE) tensile tester with a computer-based data acquisition and frame control system was used to evaluate various competitive materials. The apparatus was calibrated using national calibration standards. The tensile tester parameters were set as follows: crosshead speed of 483+/−10 mm/min and crosshead travel of 500 mm.

The laboratory conditions were maintained at 23+/−2° C. and 50 +/−5% relative humidity. Each sample was permitted to equilibrate within the testing environment for a period of at least 24 hours prior to test specimen preparation, unless the sample was a production glove or a competitive glove.

On the middle finger of each glove, a mark was made about 38 mm from the fingertip. This section was then cut from the glove using scissors. A caliper was used to measure the thickness of the glove at the finger tip. To do so, after cutting the sample from the glove, the sample was fully slid onto the test foot on the caliper. Any wrinkles in the sample were manually removed. Three measurements were taken at various positions on the finger tip and averaged.

The sample was then fully mounted on the sample mount and clamped to prevent slipping. The finger tip was powdered slightly to ensure that the material would be punctured without sticking. The crosshead was then started, and the force required to puncture the glove was recorded. The results for various competitive samples are provided below.

| Sample | Thickness (mm) | Puncture (N) |
|---|---|---|
| A | 0.078 | 16.9 |
| B | 0.056 | 10.5 |
| C | 0.097 | 16.4 |
| D | 0.058 | 10.3 |
| E | 0.054 | 11.9 |
| F | 0.066 | 8.3 |
| G | 0.080 | 12.3 |
| H | 0.050 | 8.2 |

### EXAMPLE 2

Thirty-three gloves made according to the present invention were evaluated for finger tip puncture resistance according to the procedure set forth in Example 1.

To form the experimental gloves, the formers were first heated to a temperature of about 65° C. The formers were then dipped into a plastisol containing PVC, a plasticizer, and a heat stabilizer. The plastisol was maintained at a temperature of about 65° C. The formers were dipped vertically into the plastisol for about 3 seconds. Upon removal from the removed from the plastisol, the formers were permitted to drain for about 42 seconds and rotated to a second position that formed an angle of about 80 degrees from the vertical dipping position. While being maintained at the second position, the formers were then sent through a fusion oven maintained at about 200° C. for about 5-6 minutes. The formers were then cooled to a temperature of about 100° C. using fans.

The formers were then dipped into a composition including an acrylic emulsion to form the donning layer. After drying, a bead was rolled on each glove, and the gloves were removed from the formers.

The gloves were found to have an average fingertip thickness of 0.12 mm. Also, the average force required to puncture the fingertips was 29.2 N. Thus, the gloves of the present invention were significantly more resistant to puncture in the fingertip.

### EXAMPLE 3

A simulated use in durability study was performed to evaluate the glove of the present invention. The study was designed to mimic the stresses on examination gloves in clinical situations, and is described in detail in “Performance of latex and nonlatex medical exarmination gloves during simulated use” by D. Korniewicz et al. (American Journal of Infection Control, Vol. 30, No. 2, pp. 133-138). In general, the subjects are asked to don the glove sample and perform the following tasks: (1) connect a syringe to a stopcock, turn it on and off 30 times, then disconnect the syringe using a hemostat, and repeat this procedure 10 times; (2) connect and disconnect a suction tube to a catheter 10 times; (3) wrap a blunt object (e.g. an artificial hand) with gauze and apply 2 pieces of fresh tape 3 times; and (4) rub each gloved hand with a washcloth in clean water with the following sequence: palm, each finger in a twisting motion, thumb, and back of hand. After completion of the each task, the gloves were visually inspected. If a defect was observed, the glove failed. After completion of the tasks, if no defects were observed during inspection, the gloves were subjected to the FDA water-leak test, which entails filling the glove with 1000 ml of water, suspending the glove for two minutes, and observing the glove for leaks. The location of any glove failure was noted.

The experimental glove of Example 2 was compared with a control glove commercially available under the trade name “Safeskin Clear” PVC, known to be a PVC glove formed by traditional glove formation processes, and three competitive glove samples. A sample size of 250 was used for each glove evaluated, with the following results:

| Failure | Experi- | | Competitive | Competitive | Competitive |
|---|---|---|---|---|---|
| Location | mental | Control | Sample J | Sample K | Sample L |
| Wrist | 1 | 0 | 1 | 0 | 0 |
| Palm | 1 | 0 | 0 | 0 | 0 |
| Finger | 7 | 1 | 2 | 0 | 2 |
| Fingertip | 1 | 24 | 82 | 42 | 23 |
| Total | 10 | 25 | 85 | 42 | 25 |
| Failure | 4% | 10% | 34% | 16.8% | 10% |
| rate | | | | | |
| Fingertip | 0.4% | 9.6% | 32.8% | 16.8% | 9.2% |
| failure | | | | | |
| rate | | | | | |
| Fingertip | 0.12 | 0.08 | 0.066 | 0.05 | 0.056 |
| thickness | | | | | |
| (mm) | | | | | |

The total failure rate of the control glove was 10% compare to a failure rate of 4% for the experimental glove, indicating a 60% reduction in total glove failures. Furthermore, in the fingertip, the failure rate of the control glove was 9.6%, while the failure rate of the experimental glove was only 0.4%. When compared with the competitive samples J, K, and L, the experimental glove exhibited a significant decrease in failures.

The results indicate that there is a strong correlation between failure rate and fingertip thickness. The experimental gloves formed according to the present invention had thicker tips, thereby offering increased resistance to failure.

In sum, the method of forming a glove according the present invention, and the glove formed thereby, offer significant advantages over traditional glove formation processes and gloves. By adjusting the angle of the former during gelation of the PVC plastisol, a glove is formed that has an increased thickness in the fingertips. Since the fingertip area is most prone to failure, the glove formed according to the present invention is significantly less prone to failure.

The invention may be embodied in other specific forms without departing from the scope and spirit of the inventive characteristics thereof. The present embodiments therefore are to be considered in all respects as illustrative and not restrictive, the scope of the invention being indicated by the appended claims rather than by the foregoing description, and all changes which come within the meaning and range of equivalency of the claims are therefore intended to be embraced therein.

1. A method of forming a glove having improved fingertip puncture resistance comprising:
providing a glove former pivotably attached to a chain assembly;
dipping the former into a plastisol in a first position, the first position being substantially vertical;
removing the former from the plastisol;
pivoting the former to a second position, the second position forming an angle less than 90 degrees with respect to the first position; and
maintaining the former at the second position until the plastisol forms a gel on the former.

2. The method of claim 1, wherein the second position forms an angle of from about 60 degrees to about 85 degrees with respect to the first position.

3. The method of claim 1, wherein the second position forms an angle of from about 70 degrees to about 83 degrees with respect to the first position.

4. The method of claim 1, wherein the second position forms an angle of from about 75 degrees to about 80 degrees with respect to the first position.

5. The method of claim 1, further comprising heating the former while maintaining it at the second position.

6. The method of claim 1, wherein the former is rotatably attached to the chain assembly by a former carrier, and wherein the former rotates about an axis formed by the length of the carrier.

7. The method of claim 1, further comprising cooling the gel.

8. The method of claim 1, wherein the plastisol comprises polyvinyl chloride.

9. A polyvinyl chloride glove having improved fingertip puncture resistance comprising:
a palm portion having a palm thickness; and
a plurality of fingers extending from the palm portion, each finger having a fingertip distal to the palm portion,
wherein the fingertip has a fingertip thickness substantially equal to the palm thickness.

10. The glove of claim 9, wherein the fingertip thickness is from about 0.1 mm to about 0.2 mm.

11. The glove of claim 9, wherein the fingertip thickness is from about 0.11 mm to about 0.15 mm.

12. The glove of claim 9, wherein the fingertip thickness is about 0.12 mm.

13. The glove of claim 9, formed by method comprising:
providing a glove former, the former pivotably attached to a chain assembly;
dipping the former into a polyvinyl chloride resin plastisol in a first position, the position being substantially vertical;
removing the former from the plastisol;
pivoting the former to a second position, the second position forming an angle less than 90 degrees with respect to the first position; and
maintaining the former at the second position until the plastisol gels on the former.

14. The glove of claim 13, formed by the method further comprising heating the former while maintaining it at the second position.

15. A method of determining fingertip puncture resistance in a glove comprising:
preparing a glove fingertip sample;
placing the sample onto a cylindrical sample mount;
advancing a probe toward the sample;
contacting the probe to the sample; and
measuring the force required to perforate the sample.

16. The method of claim 15, further comprising measuring the thickness of the sample.

17. The method of claim 15, further comprising applying a powder to the fingertip sample.

18. The method of claim 15, further comprising clamping the fingertip sample to the cylindrical sample mount.

19. The method of claim 15, wherein the probe is advanced toward the sample at from about 100 mm/min to about 800 mm/min.

20. The method of claim 15, wherein the probe is advanced toward the sample at from about 400 mm/min to about 600 mm/min.
